(19) United States
(12) Patent Application Publication
(10) Pub. No.: US 2015/0374206 A1
(43) Pub. Date: Dec. 31, 2015

(54) Methods and Systems for Managing Information Generated From and Transmitted To An Endoscopic System

(71) Applicant: EndoChoice, Inc., Alpharetta, GA (US)

(72) Inventors: Anat Shimony, Tzrufa (IL); Tal Davidson, Yokneam Ilit (IL); Roi Livne, Haifa (IL)

(21) Appl. No.: 14/751,835

(22) Filed: Jun. 26, 2015

Related U.S. Application Data
(60) Provisional application No. 62/017,423, filed on Jun. 26, 2014.
(60) Provisional application No. 62/017,701, filed on Jun. 26, 2014.

(51) Int. Cl. A61B 1/00

(52) U.S. Cl. CPC A61B 1/00059; A61B 1/00006; A61B 1/00016

(57) ABSTRACT

The specification discloses a method of operating an endoscope that includes a main connector at a proximal end and an insertion section extending from the main connector towards a distal end, the main connector being operatively connected with a control unit, the method including: storing operational information of one or more replaceable components of the endoscope in a first portion of a memory of the main connector; storing manufacturing information including at least one manufacturing property of the endoscope in a second portion of the memory of the main connector, wherein the first portion of the memory is logically separated from the second portion; retrieving the stored information; and conveying the retrieved information. The specification also discloses a method and system for preprocessing of imaging data in an endoscopy system to ensure that the quality of image data does not deteriorate during transmission to any external documentation systems.

### CROSS-REFERENCE TO RELATED APPLICATIONS

The present application relies on, for priority, the following United States Provisional Patent Applications, which are also herein incorporated by reference in their entirety:

U.S. Provisional Patent Application No. 62/017,423, entitled “Memory Device For An Endoscope” and filed on Jun. 26, 2014; and

U.S. Provisional Patent Application No. 62/017,701, entitled “Systems and Methods for Pre-Processing Images For User Documentation Systems” and filed on Jun. 26, 2014.

### FIELD

The present specification generally relates to the management of information that is generated by and transmitted to an endoscopic system. In particular, the present specification relates to storage and retrieval of data generated from various components of the endoscope, thus aiding in monitoring the operation of the components. Also, the present specification relates to methods for preprocessing of images in an endoscopy system before exporting them to an external documentation system.

### BACKGROUND

Endoscopes have attained great acceptance within the medical community, since they provide a means to perform procedures with minimal patient trauma, while enabling the physician to view the internal anatomy of the patient. Over the years, numerous endoscopes have been developed and categorized according to specific applications, such as cystoscopy, colonoscopy, laparoscopy, upper gastrointestinal (GI) endoscopy and others. Endoscopes may be inserted into the body's natural orifices or through an incision in the skin.

An endoscope is usually an elongated tubular shaft, rigid or flexible, having one or more video cameras or fiber optic lens assemblies at its distal end. The shaft is connected to a handle, which sometimes includes an ocular for direct viewing. Viewing is also usually possible via an external screen. Various surgical tools may be inserted through a working channel in the endoscope to perform different surgical procedures.

Endoscopes may have a front camera and a side camera to view internal organs, such as the colon, illuminators for each camera, one or more fluid injectors (“jet”) to clean the camera lens(es) and sometimes a working channel to insert surgical tools, for example, to remove polyps found in the colon. Often, endoscopes also have gas injectors to inflate a body cavity, such as the colon, into which they are inserted. The illuminators commonly used are fiber optics which transmit light, generated remotely, to the endoscope tip section. The use of light-emitting diodes (LEDs) for illumination is also known.

The various components of an endoscope assembly have a limited lifetime, and/or are subject to failures during surgeries. This is highly undesirable, especially if failure is discovered during a surgical procedure. Based on various pieces of information, such as warranty information, usage frequency and usage statistics, the lifetime of these components may be reasonably predicted. Therefore, it is essential to be able to track usage information for the various components of an endoscope thus, allowing users and/or manufacturers to understand current or potential defects, errors, or other issues. It is, however, burdensome for users to manually track usage history. For example, monitoring and storing the number of hours of use of the endoscope, or the number of hours of use of LED light sources, is an onerous task for the endoscope's operator. Additionally, such information is often required for multiple endoscopic devices deployed in a hospital environment. Still further, more than one user may have access to the endoscope, making such manual tracking even more difficult.

Current endoscopy systems provide for the ability to track cumulative usage information of replaceable components, such as light sources. One method for collecting and storing this information is through RFID tags that communicate with RF transceivers. While currently available systems enable tracking replaceable components, this information alone may be insufficient for overall understanding of potential defects, errors, or other issues. Thus, there is a need to aggregate and identify information that includes tracking of replaceable components and properties of replaceable and irreplaceable components.

In an electronic endoscopy system, the main control unit, which is used to process data from an endoscope, is generally a separate unit from the endoscope itself, which is a device that can be removably attached to the main control unit. The main control unit comprises a front panel and/or a display screen for displaying operational information with respect to an endoscopy procedure when the endoscope is in use. The display screen may be configured to display images and/or video streams received from the viewing elements of the multi-viewing element endoscope. The screen may further be operative to display a user interface for allowing a human operator to set various features of the endoscopy system.

The imaging data from the endoscopy system is exported to external documentation systems in hospitals, clinics, medical institutes which generally comprise the software programs used for management, analysis and reporting of data related to an endoscopy procedure. Many physicians also use a specialized electronic medical recording system known as an endoscopy report writer (ERW). The ERW is a computer software database that stores the text report and includes data fields that are populated by the physician.

A frequent problem faced by physicians during the transfer of data to an endoscopy report writer is the degradation in quality of data during the transmission process. Various attributes of the image/video stream are modified while data is transferred from the endoscopy system to the ERW. Often, color attributes such as hue, contrast, brightness, tone, and chroma are modified during the data transfer. In some cases, physical properties of images such as aspect ratio and resolution may also be impacted.

There are multiple methods through which the image data is transferred from endoscopy system to any user documentation system like ERW. Image data may be transferred through physical wires or via wireless transmission. Usually the transfer process involves conversion of data from a digital format to an analog format and back to a digital format, which leads to significant degradation in the quality of the image that eventually reaches the ERW. The above degradation in image quality can significantly reduce the reliability of the medical procedure and can make it difficult for a physician to accurately interpret the findings.

Several ERWs have image enhancement features such as tone mapping, color correction and normalization, contrast enhancement, noise suppression, and edge detection to improve the quality of images. However, these methods have limitations and fail to improve the image quality significantly. In addition, as there are a variety of endoscopy report writers available in the market and multiple versions of them are used in different hospital/users sites, it becomes very difficult to fine tune the images for different ERWs at different hospital/users sites.

Hence, there is need for a system and method to transfer endoscope imaging data in a more accurate and reliable manner while reporting the finding of an endoscope procedure to external documentation systems to reduce the complexity of fine-tuning the images at a hospital site.

### SUMMARY

In some embodiments, the present specification discloses a method of operating an endoscope comprising a main connector at a proximal end and an insertion section extending from the main connector towards a distal end, the main connector being operatively connected with a control unit, the method comprising: storing operational information comprising usage information of one or more replaceable components of the endoscope in a first portion of a memory of the main connector; storing manufacturing information comprising at least one manufacturing property of the endoscope in a second portion of the memory of the main connector, wherein the first portion of the memory is logically separated from the second portion of the memory; retrieving the stored information; and conveying the retrieved information.

Optionally, the method further comprises storing operational information of the control unit in a memory of the control unit.

Optionally, storing operational information comprises storing at least one of an endoscope revision number, a date of last use of the endoscope, a usage information of one or more light sources, a cumulative number of procedures conducted using the endoscope, and a cumulative number of times the endoscope device is connected to the control unit.

Still optionally, storing operational information of the control unit comprises storing at least one of the cumulative operational time of the control unit, an operation time of the control unit during an endoscopy procedure, a number of times that the control unit is connected with the endoscope.

Optionally, storing manufacturing information comprises storing at least one of a serial number of the endoscope, a type of the endoscope, and a type of video captured by the endoscope.

Optionally, storing operational information further comprises collecting usage information from at least one replaceable component of the endoscope device. Still optionally, collecting usage information comprises using Radio Frequency (RF) communication to obtain the usage information recorded in an RFID tag coupled with the replaceable component.

Optionally, conveying the retrieved information comprises displaying the retrieved information on a monitor connected to at least one of the control unit and the endoscope. Still optionally, conveying the retrieved information comprises communicating the retrieved information to a server operatively connected to the endoscope.

In some embodiments, the present specification discloses a method of operating an endoscope comprising a main connector at a proximal end and an insertion section extending from the main connector towards a distal end, the main connector being operatively connected with a control unit, the method comprising: storing operational information of the control unit in a first memory device; storing operational information comprising usage information of one or more replaceable components of the endoscope in a first portion of a second memory device; storing manufacturing information comprising at least one manufacturing property of the endoscope in a second part of the second memory device; retrieving the stored information; and, conveying the retrieved information.

Optionally, storing operational information of the control unit comprises storing at least one of the cumulative operational time of the control unit, an operation time of the control unit during an endoscopy procedure, a number of times that the control unit is connected with the endoscope. Still optionally, the operational information comprising usage information of one or more replaceable components of the endoscope comprises storing at least one of an endoscope revision number, a date of last use of the endoscope, a usage information of one or more light sources, a cumulative number of procedures conducted using the endoscope, and a cumulative number of times the endoscope device is connected to the control unit. Still optionally, storing manufacturing information of the endoscope device comprises storing at least one of a serial number of the endoscope, a type of the endoscope, and a type of video captured by the endoscope. Still optionally, storing operational information comprising usage information of one or more replaceable components of the endoscope further comprises collecting operational information from at least one replaceable component of the endoscope device. Optionally, collecting usage information comprises using Radio Frequency (RF) communication to obtain the usage information recorded in an RFID tag coupled with the replaceable component.

Still optionally, conveying retrieved information comprises displaying retrieved information on a monitor connected to at least one of the control unit and the endoscope device. Still optionally, conveying the retrieved information comprises communicating the retrieved information to a server operatively connected to the endoscope.

Optionally, the endoscope is connected to the control unit, the control unit is adapted to detect the connection and cause a memory counter to be updated, wherein said memory counter is configured to track a cumulative number of times the endoscope is plugged to the control unit.

Optionally, the endoscope is connected to the control unit, the control unit is adapted to detect the connection and cause a memory counter to be updated to a new date, wherein said memory counter is configured to track a last usage date of the endoscope.

Optionally, the endoscope further comprises a plurality of illuminators wherein, when at least one of said plurality of illuminators is switched on, the control unit is adapted to send a signal to a memory counter, wherein said memory counter is configured to track a number of endoscopy procedures performed and a cumulative number of times each of said plurality of illuminators were switched on or off.

Optionally, the endoscope further comprises a plurality of illuminators wherein, when at least one of said plurality of illuminators is switched off, the control unit is adapted to send a signal to a memory counter, wherein said memory counter is configured to track a number of endoscopy procedures performed and a cumulative number of times each of said plurality of illuminators were switched on or off.

Optionally, when the endoscope is disconnected from the control unit, the control unit is adapted to detect the disconnection and cause a memory counter to be updated, wherein said memory counter is configured to track a total duration for which the endoscope remained plugged into the control unit.

Optionally, the control unit is adapted to generate at least one of an average duration for a single procedure over a predefined period of time, a longest duration for single procedure, a shortest duration for a single procedure, and an average duration of use for a single procedure on a per physician basis.

In some embodiments, the present specification discloses a method for pre-processing image data captured by an endoscopy system to compensate for image data degradation during data transfer to an external documentation system, the method comprising: transmitting a first image data from the endoscopy system to the external documentation system, wherein the first image data is modified to second image data in the external documentation system; transmitting said second image data received by the external documentation system back to the endoscopy system via a network connection; comparing the first image data transmitted from the endoscopy system with the second image data received back by the endoscopy system to determine a first mathematical function corresponding to one or more changes in the second image data relative to the first image data; and, generating a second mathematical function based upon the first mathematical function; applying the second mathematical function to the first image data to create a third image data; and transmitting the third image data from the endoscopy system to the external documentation system.

Optionally, the endoscopy system comprises a control unit operatively connected with an endoscope and wherein the control unit pre-processes the first image data captured by the endoscope.

Optionally, the external documentation system comprises an endoscopy report writing software.

Optionally, the first image data comprises continuous video streaming data. Still optionally, the first image data is characterized by at least one of color data, hue data, contrast data and brightness data. Still optionally, the first image data comprises an entire video stream generated in a course of an endoscopy procedure, wherein the second image data comprises fewer frames than the first image data, and wherein the third image data comprises substantially a same number of frames as the first image data. Still optionally, the first image data comprises a plurality of frames generated in a course of an endoscopy procedure, wherein the second image data comprises less than 70% of the plurality of frames in the first image data, and wherein the third image data comprises approximately 90%-110% of the plurality of frames in the first image data.

Optionally, the second mathematical function is an inverse of the first mathematical function. Still optionally, the first mathematical function causes at least one color, black level, sharpness, tone, chroma, hue, contrast and brightness of the first image data to increase in a range of 5% to 30% and wherein the second mathematical function causes at least one color, black level, sharpness, tone, chroma, hue, contrast and brightness of the first image data to decrease in a range of 5% to 35%. Still optionally, the first mathematical function causes at least one color, black level, sharpness, tone, chroma, hue, contrast and brightness of the first image data to decrease in a range of 5% to 30% and wherein the second mathematical function causes at least one color, black level, sharpness, tone, chroma, hue, contrast and brightness of the first image data to increase in a range of 5% to 35%.

Optionally, the second mathematical function is determined once and is applied to all subsequently generated first image data captured by the endoscopy system throughout a duration of an endoscopy procedure.

In some embodiments, the present specification discloses a system for pre-processing image data captured by an endoscopy system before transmission from the endoscopy system to an external documentation system for compensating for image data degradation during data transfer, the system comprising: transmitting the image data from the endoscopy system to the external documentation system; a feedback system for transmitting the image data received by the external documentation system back to the endoscopy system; comparing the image data transmitted by the endoscopy system with the image data received by the endoscopy system via the feedback control system to determine a first mathematical function corresponding to one or more changes in the transmitted image data and the received image data; generating a second mathematical function based on the first mathematical function; applying the second mathematical function to the image data captured by the endoscopy system to create a second image data; and transmitting the second image data from the endoscopy system to the external documentation system.

Optionally, the endoscopy system comprises a main control unit operatively connected with an endoscope, the main control unit pre-processing the image data captured by the endoscope.

Optionally, the external documentation system comprises an endoscopy report writing software.

Still optionally, the image data comprises continuous video streaming data. Still optionally, the image data comprises information about at least one of color, hue, contrast and brightness of the image captured by the endoscopy system. Still optionally, the image data comprises information about at least one physical property of the image captured by the endoscopy system. Still optionally, the image data transmission is performed via one of physical wires, a wireless network, and manual submission. Still optionally, the image data transmission comprises converting digital image data to analog image data.

Optionally, the mathematical function is determined and an inverse of the determined mathematical function is applied to the image data captured by the endoscopy system continuously throughout the duration of the image data transmission. Still optionally, the mathematical function is determined only once and an inverse of the mathematical function is applied to all subsequent image data captured by the endoscopy system throughout the duration of the image data transmission.

The aforementioned and other embodiments of the present invention shall be described in greater depth in the drawings and detailed description provided below.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1 shows a semi-pictorial view of an endoscopy system, according to some embodiments;

FIG. 2A is a block diagram showing components of a control unit of an endoscope system, according to some embodiments;

FIG. 2B is an exemplary flow process diagram illustrating the generation of operational data or information related to an endoscope, according to some embodiments;

FIG. 2C is an exemplary flow process diagram illustrating the generation of operational data or information related to the control unit of FIG. 2A, according to some embodiments;

FIG. 3 is a flow chart illustrating an exemplary process executed within a control unit to access data or information stored in a memory device located at a main connector of the endoscope, according to some embodiments;

FIG. 4 illustrates an exemplary flow of a process involving data storage to and retrieval from the memory device located at the main connector of the endoscope;

FIG. 5 illustrates another exemplary flow of a process involving data storage to and retrieval from either the memory device located at the main connector of the endoscope or at a memory device located at the control unit of the endoscope system;

FIG. 6 illustrates a block diagram of an endoscopy system and a hospital data management system according to some embodiments;

FIG. 7 illustrates a method for pre-processing image and/or video data in an endoscopy system according to some embodiments;

FIG. 8A illustrates a first step of the image pre-processing method of FIG. 7;

FIG. 8B illustrates a second step of the image pre-processing method of FIG. 7;

FIG. 8C illustrates a third step of the image pre-processing method of 7;

FIG. 9A illustrates an exemplary image of a lumen of a human body captured through an endoscopy procedure;

FIG. 9B illustrates an exemplary image received by an endoscopy report writer (ERW) upon transmission of the original image illustrated in FIG. 9A from an endoscopy system;

FIG. 9C illustrates an exemplary modulated image before transmission from the endoscopy system to the ERW; and

FIG. 9D represents a final image received by the ERW after transmission from the endoscopy system.

### DETAILED DESCRIPTION

In an embodiment, the present specification discloses a method of storing data generated from various components of an endoscope, thus aiding in monitoring the operation of the components and conveying the data to an operator when required. The method comprises storing operational information concerning operation of the endoscope device, in a first portion of a memory device; storing manufacturing information concerning at least one manufacturing property of the endoscope device in a second portion of the memory device; retrieving stored information; and conveying the retrieved information.

In an embodiment, the present specification also discloses a method and system for pre-processing images in an endoscopy system before exporting the images to an external user documentation system. In an embodiment, the external documentation system comprises a specialized electronic medical record, known as endoscopy report writer (ERW), which is a computer database software that stores and generates a text report and other information related to an endoscopy procedure.

Transmission of imaging data/video streams from an endoscopy system to an external documentation system like ERW generally involves some degradation of data quality. The degradation of data quality leads to several changes in the attributes of the image/video stream received at the ERW which can make the report unreliable and can potentially cause false diagnosis of medical conditions. In an embodiment of the present specification, the changes in attributes of image/video data that might occur during the transmission process are estimated beforehand and the images are accordingly modified before transmission to ensure that the external documentation system receives the actual images. In one embodiment, a feedback control system is disclosed. The imaging data received by the external documentation system is sent back to the endoscopy system for comparison with the original imaging data to estimate a mathematical function indicative of the changes in the data. In one embodiment, the inverse of this mathematical function is applied to the imaging data before exporting the same from the endoscopy system to any external documentation system to offset the impact of changes that occur during the transmission process.

In one embodiment, the feedback control system is used only during the initial set-up or calibration phase to derive an estimated constant mathematical function and subsequently all data is transmitted after modification in accordance with the estimated mathematical function. In another embodiment, the feedback control system operates continuously between the external documentation system and the endoscopy system and the mathematical function is generated dynamically in real time before transmitting any data. In one embodiment, the feedback control system is used only at pre-defined time intervals to recalibrate the mathematical function F(X) such that if there is any change in the mathematical function F(X) with time, the same is accounted for in the recalibrated function. In one embodiment, the pre-defined time interval could be daily, or weekly or monthly as per the system requirement.

In practical scenarios, the mathematical function indicative of changes in the data may be different for each specific pair of an endoscopy system and corresponding user documentation system, which may also depend on the medium of transmission. In one embodiment, a separate feedback mechanism is used for each specific pair of an endoscopy system and corresponding user documentation system to estimate the corresponding mathematical function which is then used for modulating data transmitted between that specific endoscopy system and user documentation system pair.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

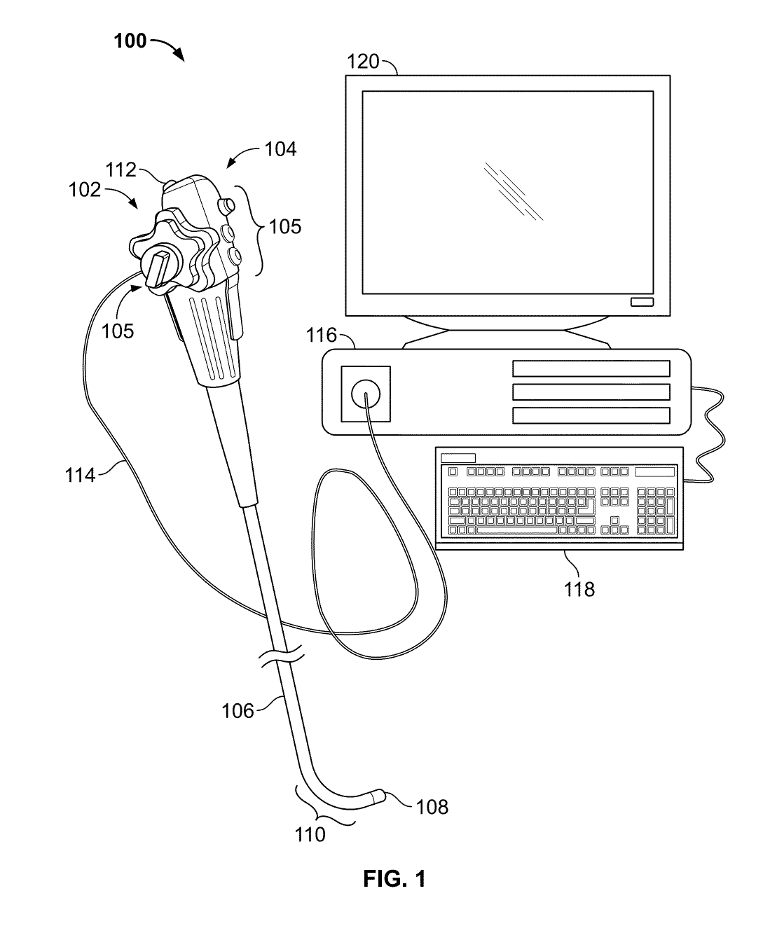

Reference is now made to FIG. 1 which shows a pictorial view of a multi-viewing element endoscopy system 100. System 100 includes a multi-viewing element endoscope 102. The multi-viewing element endoscope 102 includes a handle 104, from which an elongated shaft 106 emerges. The elongated shaft 106 terminates with a tip section 108 which can be turned by a bending section 110. The handle 104 is used to maneuver elongated the shaft 106 within a body cavity. The handle 104 may include one or more knobs and/or switches (or buttons or valves) 105 that control the bending section 110 as well as functions such as fluid injection and suction, and toggling between multi-viewing elements of the tip section 108. The handle 104 further includes a service or working channel opening 112 through which surgical tools may be inserted. In alternative embodiments, the location of each component on the handle 104 may be other than the illustrated locations.

The tip section 108 includes multiple viewing elements. In accordance with an embodiment, the tip section 108 includes a front viewing element and one or two side viewing elements. In another embodiment, the tip section 108 may include only a front viewing element. Each of the viewing elements includes a lens assembly mounted on an image sensor such as a Charge Coupled Device (CCD) or a Complementary Metal Oxide Semiconductor (CMOS) image sensor. In various embodiments, the tip section 108 also includes one or more discrete illuminators associated with each of the viewing elements (front and one or two side viewing elements) to illuminate the field of views of the respective viewing elements. In embodiments, the discrete illuminators include a light-emitting diode (LED), which may be a white light LED, an infrared light LED, a near infrared light LED, an ultraviolet light LED, or any other LED.

In addition, the tip section 108 includes at least one service or working channel exit point. In accordance with an embodiment, the tip section 108 includes a front service or working channel exit point and at least one side service channel exit point. In another embodiment, the tip section 108 includes two front service or working channel exit points.

A utility cable 114 connects the handle 104 to a control unit 116. The utility cable 114 includes therein one or more fluid channels and one or more electrical channels. The electrical channel(s) includes at least one data cable to receive image and/or video signals from the front and side-viewing elements, as well as at least one power cable to provide electrical power to the viewing elements and to the associated discrete illuminators.

The control unit 116 governs power transmission to the tip section 108, such as for the viewing elements and illuminators. The control unit 116 further controls one or more gas, fluid, liquid and/or suction pumps that supply corresponding functionalities to the endoscope 102. One or more input devices, such as a keyboard 118, a computer, a touch screen and the like, are connected to the control unit 116 for the purpose of human interaction with the control unit 116. In another configuration (not shown), an input device, such as a keyboard, or a touch screen, is integrated with the control unit 116.

A display 120 is connected to the control unit 116, and configured to display images and/or video streams received from the viewing elements of the endoscope 102. The display 120 is operative to provide a user interface (which is touch enabled, in one embodiment) to allow a human operator to set various features of the system 100. The display 120 may further be a multi monitors display.

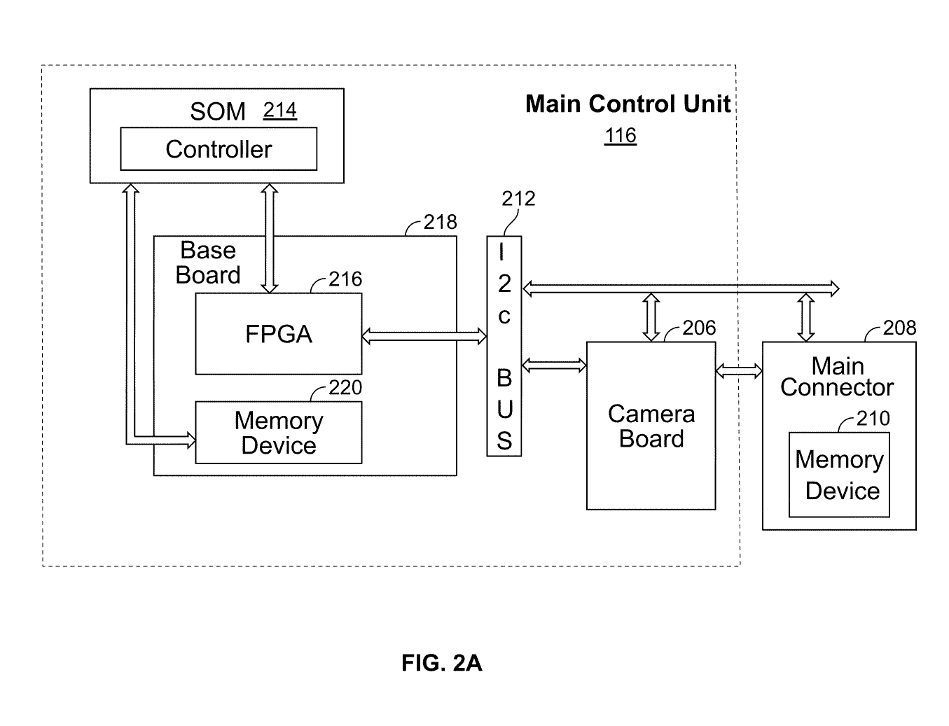

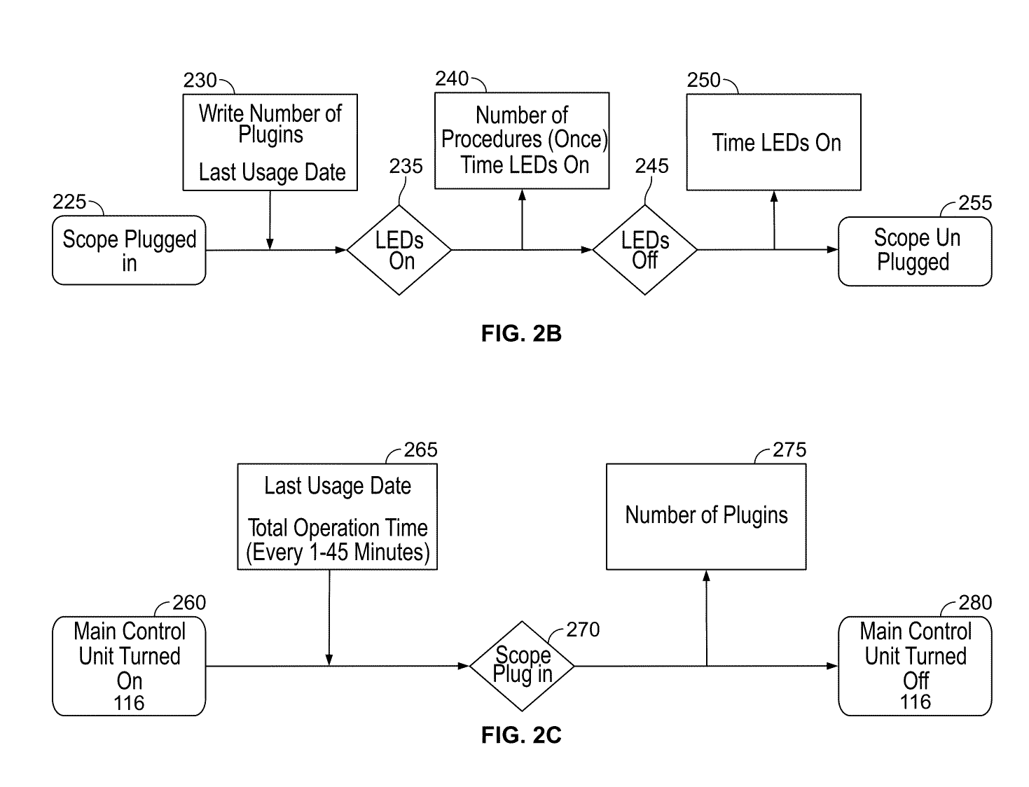

Reference is now made to FIGS. 1 and 2A. FIG. 2A is a block diagram showing components of the control unit 116 and a main connector 208 of the endoscope 102. The endoscope system 100 includes the control unit 116, hereinafter also referred to as the ‘main control unit’ (MCU) and the endoscope 102 that are operatively connected through the main connector 208. The term “endoscope” as referred to herein may refer to colonoscopes, gastroscopes, bronchoscopes or any instrument used to examine an interior of a hollow organ or cavity of a body.

In some embodiments, the endoscope 102 includes the main connector 208 at a proximal end (which connects the utility or umbilical cable 114 to the main control unit 116) and an insertion section, comprising sections such as the elongated shaft 106 and the bending section 110, extending from the proximal end towards a distal end that terminates into the tip section 108. At the distal end, the tip section 108 includes a tip cover (that is a multi-component tip cover in some embodiments) protecting internal components of the tip section 108.

In various embodiments, the internal components of the tip section 108 include an electronic circuit board assembly and a fluid-channeling component. The electronic circuit board receives signals from a camera board 206 and transmits appropriate commands to control power supply to light sources, such as the illuminators, and to control operation of the viewing elements. The camera board 206 in turn receives video signals generated by the image sensors of the viewing elements, and also various remote commands from the endoscope 102. In an embodiment, the main connector 208 includes a memory device 210. The memory device 210 is a non-volatile memory, such as but not limited to an EEPROM. In various embodiments, the memory device 210 is divided in two parts. In various embodiments, the division is a functional division. A first part of the memory device 210 comprises operational information concerning operation of the endoscope 102. The operational information is typically variable and varies or is modified with each operation of the endoscope 102. This includes information pertaining to operation of replaceable components within the endoscope to track their usage, and accurately determine potential need to replace them.

In an embodiment, Radio Frequency (RF) tags located within various replaceable components of the endoscope receive the operational information through corresponding sensors and send this information to store in the memory device 210, using RF communication. In alternative embodiments, other known types of communication methods, such as wireless (Bluetooth) or wired, are used to retrieve information and store it in the memory device 210.

In accordance with various embodiments, the operational data or information stored in the first part of the memory device 210 comprises a plurality of data or information, such as, but not limited to service, repair or maintenance information and/or information related to operational parameters that change over the period of operation of the endoscope.

Service, repair or maintenance information may include information such as, but not limited to, the date of service, name and/or code of the technician responsible for the service, type and/or code of repair, service or maintenance activity performed, cumulative number of servicing cycles performed. It should be appreciated that the service, repair or maintenance information is related to future or prospective periodic service recommended to be performed (optionally and/or mandatorily) on the endoscope and/or related to past services, repairs and maintenance activity performed on the endoscope in the past. In one embodiment, the service, repair or maintenance information related to the future or prospective service(s) is stored at the time of manufacturing of the endoscope.

Information related to operational parameters that change over the period of operation of the endoscope may include information such as, but not limited to, a date of last use of the endoscope, an operational information of the one or more light sources, such as the illuminators, a cumulative number of procedures conducted using the endoscope, a cumulative number of times the endoscope device is plugged to the control unit, an average time of plugging-in of the endoscope to the control unit. In one embodiment, the operational information related to the one or more illuminators comprises data or information such as cumulative number of times the illuminators were switched on and the duration of time the illuminators stayed on.

FIG. 2B illustrates an exemplary flow process of generating operational data or information related to the endoscope. As shown, at step 225, when the endoscope is plugged into or connected to the control unit 116 via the main connector 208, the control unit detects the connection through, for example, a conventional switch or toggle. When the control unit detects a connection, a memory counter that tracks the cumulative number of times the endoscope is plugged to the control unit is updated and also the last usage date of the endoscope is updated to the most current date (230). When the control unit is activated and causes the illuminators to be switched on (235), the control unit sends a corresponding signal to a memory counter for tracking the number of endoscopy procedures performed and the cumulative number of times the illuminators were switched on are updated (that is their counts are increased) (240). Once the control unit is activated to cause the illuminators to be switched off (245), the memory counter for tracking the total duration of time the illuminators stayed switched on is updated (250). Finally, when the control unit detects that the endoscope has been unplugged from the control unit 116 (255), the control unit sends another signal to another counter process that provides the total duration for which the endoscope remained plugged in and from which a plurality of additional statistics can be generated, such as average duration (for a single procedure) over a predefined period of time, longest duration for single procedure, shortest duration for a single procedure, and average duration of use (for a single procedure) on a per physician basis. In all cases, the memory counter may be positioned within, and part of, the control unit or in data communication with the control unit.

A second part of the memory device 210 comprises a plurality of manufacturing data or information concerning the endoscope. This information is typically fixed or non-variable and pertains to the manufacturing properties of the endoscope. Such manufacturing information includes data or information such as, but is not limited to, a serial number of the endoscope, a type of endoscope (for example, bronchoscope, colonoscope, gastroscope, or any other), a revision number of the endoscope, a type of video (for example, PAL, HD, NTSC, or any other) captured by the endoscope, or any other fixed or manufacturing-related data as would be evident to persons of ordinary skill in the art.

In various embodiments, the data or information related to manufacturing and/or service, repair and maintenance is stored by a technician during manufacturing and/or servicing activity of the endoscope. Information related to operational parameters is accumulated during use or operation of the endoscope. Also, it should be appreciated that the memory device 210 is functionally divided into the first and second parts due to a difference between the amount of operational and manufacturing data or information traffic (read and write). In one embodiment, the second portion of the memory device comprises read only memory that has a first access bus or data path while the first portion of the memory device comprises random access memory that has a second access bus or data path that is independent of the first access bus or data path. In another embodiment, the first and second portion of the memory device are the same type of memory but are logically or structurally divided, each having its own independent access bus or data path. The functional division of the memory device 210 into the two parts minimizes errors while accessing the manufacturing data or information.

In various embodiments the electrical channel that connects the handle 104 and the utility cable 114 includes an Inter-Integrated Circuit (I2C) bus 212 that connects various electronic components of the control unit 116. The I2C is a multi-master, multi-slave, single-ended, serial computer bus. It is typically used for attaching lower-speed peripheral ICs to processors and microcontrollers.

A System on Module (SOM) 214 within the control unit 116 provides an interface to input devices such as keyboard and mouse. SOM 214 is located within the control unit 116 and interfaces with various components of a base board 218 including a field-programmable gate array (FPGA) 216. FPGA 216 is a local processor that performs video interpolation and on-screen display overlay. The data or information stored in the memory device 210 is communicated to or accessed by the control unit 116 when the main connector 208 is connected to the control unit 116 via the utility cable 114, for example. The stored data or information is read by the SOM 214 via the camera board 206 and the FPGA 216. The SOM 214, in various embodiments, runs internal counters which, once in a pre-defined time steps (such as, for example, 0.01 to 5 minutes), check if the main connector 208 is still connected to the control unit 116 and write and/or read the plurality of data or information to and/or from the memory device 210. In accordance with an embodiment, the camera board 206 and the FPGA 216 control the illuminators and also track or sense operational information related to the illuminators, such as, but not limited to, when the illuminators were switched on, the cumulative number of times the illuminators were switched on and the duration for which these remain switched on.

In various embodiments, the base board 218 also includes a memory device 220. The memory device 220 is a non-volatile memory, such as but not limited to an EEPROM. In various embodiments, the memory device 220 is configured to store various types of information concerning operation of the control unit 116. The information stored in the memory device 220 comprises data or information, such as, but not limited to service, repair or maintenance information related to the endoscope; manufacturing information including data; and/or operational information related to the control unit.

In embodiments, service, repair or maintenance information related to the endoscope may include information such as, but not limited to, the date of service, name and/or code of the technician responsible for the service, type and/or code of repair, service or maintenance activity performed, cumulative number of servicing cycles performed. It should be appreciated that the service, repair or maintenance information is related to future or prospective periodic service recommended to be performed (optionally and/or mandatorily) on the endoscope and/or related to past services, repairs and maintenance activity performed on the endoscope in the past. In one embodiment, the service, repair or maintenance information related to the future or prospective service(s) is stored at the time of manufacturing of the endoscope.

In embodiments, manufacturing information may include data or information such as, but not limited to, a serial number of the endoscope, a type of endoscope (for example, bronchoscope, colonoscope, gastroscope, or any other), a revision number of the endoscope, a type of video (for example, PAL, HD, NTSC, or any other) captured by the endoscope, or any other fixed or manufacturing-related data as would be evident to persons of ordinary skill in the art.

In embodiments, operational information related to the control unit 116, may include information such as, but not limited to, data pertaining to the cumulative time that the control unit 116 has been in operation, a time and/or date of operation of the control unit 116 during an endoscopy procedure, a number of times that the control unit 116 has been plugged in with the endoscope, last date of usage of the endoscope and/or the control unit 116 or any other data concerning operation of the control unit 116.

FIG. 2C illustrates an exemplary flow process of generating operational data or information related to the control unit 116. As shown, at step 260, when the control unit 116 is switched on, the SOM 214 triggers an internal counter to update the last usage date of the control unit 116 to the current usage date and also begins tracking a total time duration of operation of the control unit 116, at step 265. Next, at step 270, when the endoscope is plugged into the control unit 116 an internal counter tracking the cumulative number of endoscope plug-ins is accordingly updated at step 275. At step 280 when the control unit 116 is switched off, the total time duration of operation of the control unit 116 is captured.

The plurality of data or information can be accessed or retrieved from the memory devices 210 and 220 and may be displayed on display units, such as the display 120, connected to the control unit 116, or any other display. The control unit 116 may have a network interface to allow it to communicate over an external network with a remote server. In various embodiments, the retrieved or accessed data or information is downloaded to remote computers to track, monitor, and analyze the endoscope system 100. For example, in case there is a complaint by the endoscope device's user about the illuminators in the distal tip 108, a technical person could analyze the endoscope 102 in light of the data or information saved in one or both of the memory devices 210 and 220. An informed analysis with this data or information helps expedite repair or redressal of any issue of the system 100 and its components.

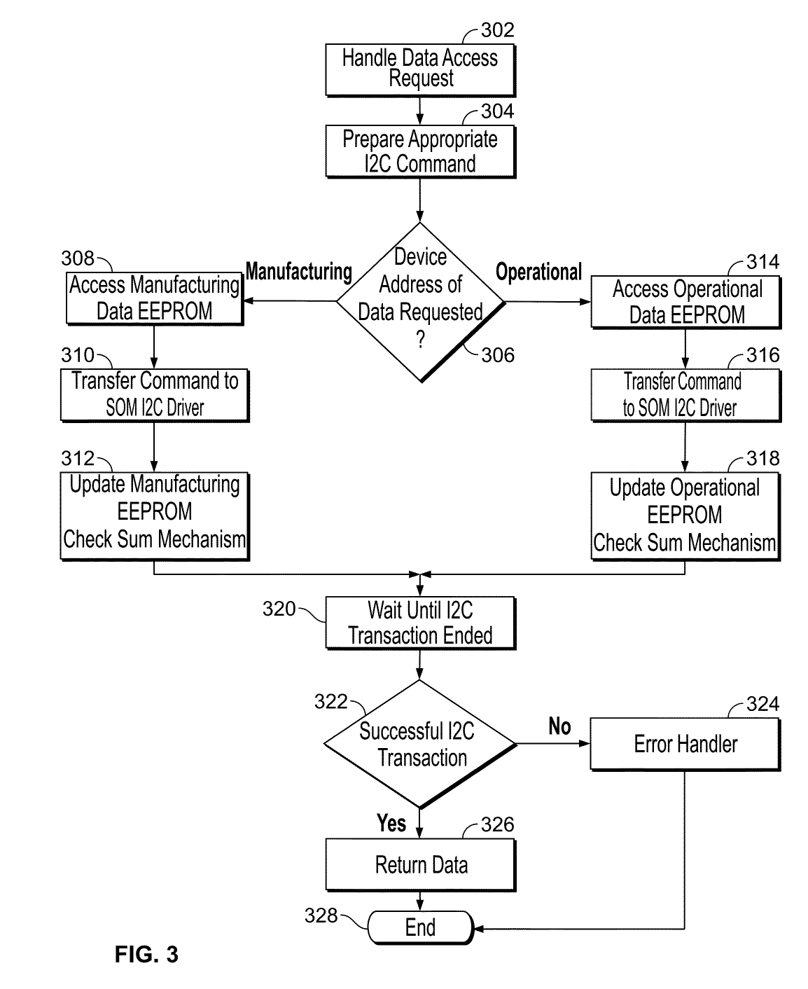

FIG. 3 illustrates an exemplary flow of a process involving data retrieval or access from the memory device 210 of FIG. 2A. Referring to FIGS. 1, 2A and 3, at step 302, the control unit 116 handles a request received to access data. The request may be provided through the user interface connected to control unit 116, or remotely over a network. At step 304, the control unit 116 subsequently prepares an appropriate I2C command to transfer over the I2C bus 212. At step 306, a check is performed, by associated software or programmatic instructions residing in the SOM 214 within the control unit 116, to determine whether the request for data pertains to operational data or manufacturing data, based on the address or location of the device from which the data is requested. If it is determined that manufacturing data is requested, then at step 308, the control unit 116 accesses the portion of the memory device 210 (that is, the second part of the memory device 210) that contains manufacturing data. At step 310, a command is transferred to the I2C driver of the SOM 214. At step 312, a checksum of the portion of memory device 210 (that is, the second part of the memory device 210) that contains manufacturing information is updated.

However, if at step 306, it is determined that the request is for operational data, then at step 314, the portion of memory device 210 (that is, the first part of the memory device 210) that contains operational data is accessed. At step 316, a command is transferred to the I2C driver of the SOM 214. At step 318, a checksum of the portion of memory device 210 (that is, the first part of the memory device 210) that contains operational data or information is updated.

At step 320, the control unit 116 waits until the I2C transaction ends and at step 322, the control unit 116 verifies whether the I2C transaction is completed successfully. If not, at step 324 an error is generated and an error handling process is initiated, followed by ending the process at step 328. In an embodiment, the error handling process includes a process that is employed to inform of a detected failure. However, if at step 322, it is found that the transaction has successfully completed, at step 326 the control unit 116 returns the requested data and ends the process at step 328. The requested data may be displayed on display units connected to the control unit 116, or communicated to a remote computer.

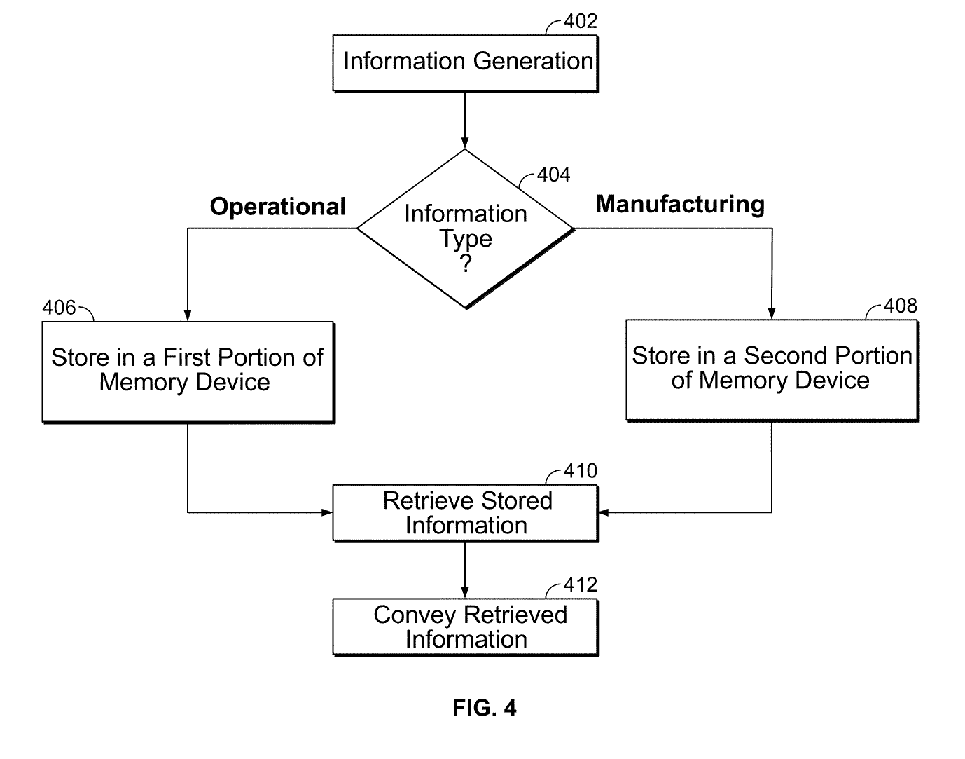

FIG. 4 illustrates another exemplary flow of a process involving data storage and retrieval from the memory device 210 of FIG. 2A. Referring to FIGS. 2A and 4, at step 402, data or information is generated. The information includes data pertaining to an endoscope device. The information is forwarded to an appropriate portion (the first part or the second part) of the memory device 210, at step 404, based on the type of information that is generated. At step 406, information pertaining to the operational data of the endoscope device is stored in the first part of the memory device 210. Alternatively, at step 408, information pertaining to manufacturing data of the endoscope device is stored in the second part of the memory device 210. Subsequently, once a request for information is made, at step 410, the requested information is retrieved from the appropriate part of the memory device 210. At step 412, the retrieved information is conveyed to its requestor through available means, such as by displaying on display units connected to the control unit 116 or communicated to a remote computer.

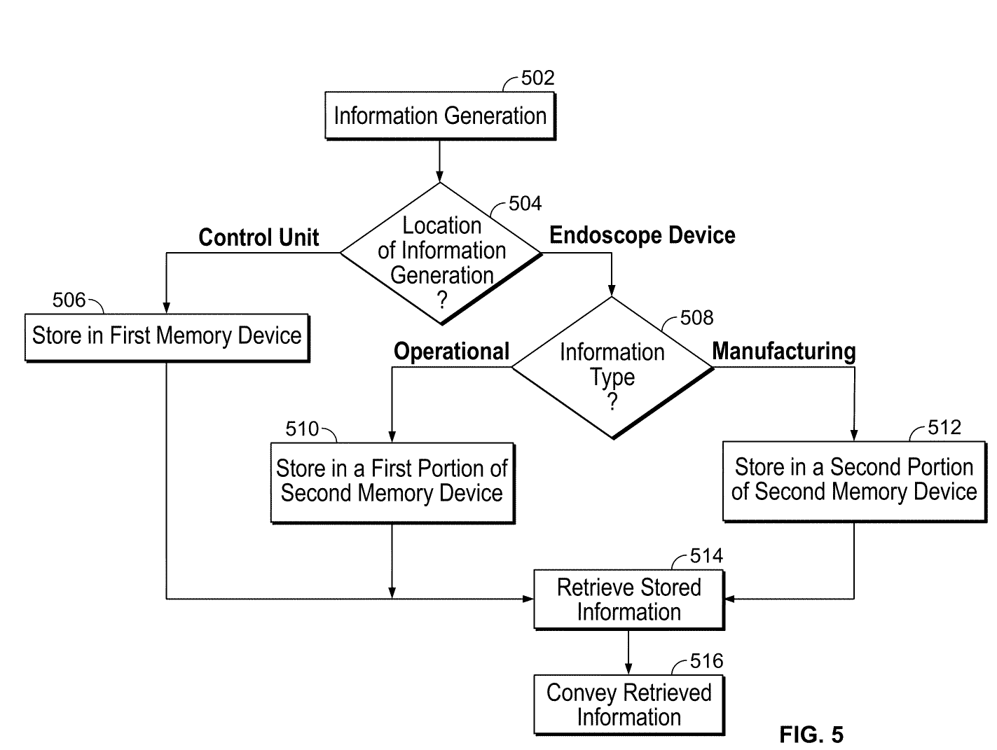

FIG. 5 illustrates another exemplary flow of a process involving data storage and retrieval from either of the memory devices 210 or 220 of FIG. 2A. Referring to FIGS. 1, 2A and 5, at step 502, information is generated as a result of manufacturing or operation of the main control unit 116 or the endoscope 102. At step 504, based on the type (that is, manufacturing or operational data or information) and the location or source (that is, the control unit 116 or the endoscope 102) of the information, the corresponding data is forwarded to either the memory device 210 or the memory device 220. At step 506, if the information relates to the control unit 116, the data is stored in a first memory device, which corresponds to the memory device 220 located within the control unit 116. If, however, the information is generated within the endoscope device 102, at step 508, the information is forwarded to an appropriate portion of a second memory, such as the memory device 210, based on the type of information that is generated. At step 510, information pertaining to operational data of the endoscope device 102 is stored in a first portion of the second memory. Alternatively, at step 512, information pertaining to manufacturing data of the endoscope device 102 is stored in a second portion of the second memory. Subsequently, once a request for information is made, at step 514, the requested information is retrieved from either the first or the appropriate portion of the second memory. At step 516, the retrieved information is conveyed to a requestor through available means, such as by displaying on display units connected to the control unit 116 or communicated to a remote computer.

Thus, various embodiments of the specification enable tracking and monitoring of various components of the endoscope device individually and independently of the entire endoscope assembly. The data is organized and conveyed for repair and maintenance purposes, and is also used to generate alerts for the users about potential maintenance and /or repair requirements.

FIG. 6 illustrates a block diagram of endoscopy and hospital data management systems in accordance with an embodiment of the present specification. As shown in FIG. 6, the endoscopy system 601 comprises an endoscope 610 and a main control unit 602 which, in an embodiment, contains or implements the controls required for displaying images of internal organs captured by the endoscope 610 on at least one display device. In one embodiment, the main control unit 602 governs power transmission to the endoscope's tip section 604, such as for the tip section's viewing elements 605 and associated illuminators. In one embodiment, the main control unit 602 may further control one or more fluid, liquid and/or suction pump(s) which supply corresponding functionalities to the endoscope 610. In an embodiment, one or more input devices, such as a keyboard, a touch screen, at least one monitor (not shown) and the like may be connected to main control unit 602 for the purpose of human interaction with the main control unit 602. In an embodiment, the main control unit 602 also comprises a front panel and/or a display screen for displaying operation information concerning an endoscopy procedure when the endoscope 610 is in use. The display screen is configured to display images and/or video streams received from the viewing elements 605 of the multi-viewing element endoscope 610. The screen is further operative to display a user interface for allowing a human operator to set various features of the endoscopy system 601.

Optionally, the video streams received from the different viewing elements 605 of the multi-viewing element endoscope 610 are displayed separately on at least one monitor by uploading information from the main control unit 602, either side-by-side or interchangeably (namely, the operator may switch between views from the different viewing elements manually). Alternatively, these video streams are processed by the main control unit 602 to combine them into a single, panoramic video frame, based on an overlap between fields of view of the viewing elements 605. In an embodiment, two or more displays are connected to the main control unit 602, each for displaying a video stream from a different viewing element of the multi-viewing element endoscope 610. In an embodiment, the endoscopy system 601 comprises a handle 603 which contains the means (such as, actuatable buttons and/or switches) through which a physician can perform the endoscopy procedure and control various functionalities of the endoscopy system 601. As shown in FIG. 6, the handle 603 and the main control unit 602 are in data communication with the endoscope tip section 604, which in an embodiment, contains a plurality of viewing elements 605 located on the endoscope tip section 604 that capture the imaging information from inside the patient's body and sends it to main control unit 602 for display and further processing.

In an embodiment, the imaging data captured by the endoscopy system 601 is transferred to an external system such as the hospital, clinic or medical institute data management system 606 shown in FIG. 6. In an embodiment, the hospital data management system 606 is a typical computer software program used in hospitals to manage various functions including documentation and reporting of medical procedures. In one embodiment, the hospital data management system 606 comprises a documentation system such as an endoscopy report writer (ERW) 607, which is a computer database software specifically used for management, storage and reporting of information pertaining to endoscopy procedures. As shown in FIG. 6, the endoscopy system 601 and the hospital data management system 606 are in data communication with each other through a data link 608.

Referring to FIG. 6, there are multiple methods via which the imaging data is transferred from the endoscopy system 601 to the hospital data management system 606. In one embodiment, the transfer is done through physical wires through commonly used data transfer standards. In another embodiment, the endoscopy system 601 and the data management system 606 are configured for wireless transmission of data between the two systems.

In one embodiment, the imaging data displayed on the display screens in endoscopy system 601 is first converted from digital to analog format and subsequently it is transferred to the data management system 606 through physical wires, where it is again converted from analog to digital format. Depending on the method of data transfer between the endoscopy system 601 and the data management system 606, there is generally some degradation in the quality of image during the transmission of data.

In an embodiment of the present specification, a data link which is a two-way data communication channel 608 is disclosed such that the imaging data received by the endoscopy report writer 607 is sent back to the main control unit 602 through a feedback control loop. In an embodiment, the main control unit 602 compares original imaging data with the imaging data received through the feedback control loop to evaluate the changes that occur during the transmission process. In one embodiment, the main control unit 602 subsequently modulates the imaging data to offset the impact of these changes, due to transmission related quality degradation, beforehand such that the image/video stream received by the endoscopy report writer 607 is same as the actual or original imaging data captured by the viewing elements 605 of the endoscopy system 601. In one embodiment, the main control unit 602 contains predefined definitions and/or algorithms to estimate differences between various parameters of an actual or original image with those of the image received through the feedback control loop and accordingly modulates the actual image to offset the impact of these estimated differences before transmitting the imaging data.

One of ordinary skill in the art would appreciate that there may be multiple ways to implement the feedback control loop or system. In one embodiment, the feedback control loop is used only during the initial set-up phase to estimate the changes in imaging data that occur during the data transmission process and subsequently, all imaging data is modulated before transmission to ensure that the impact of transmission process is pre-accounted for. In one embodiment, the feedback control is implemented manually by capturing the data received by the endoscopy report writer 607 on a memory device and providing this information to the endoscopy system 601 for comparison. In another embodiment, the feedback control loop is operated during the entire data transmission process such that the imaging and/or video stream received by the endoscopy report writer 607 is continuously transmitted to the main control unit 602 to estimate the changes that occur in the transmission process and accordingly modulate the transmitted imaging and/or video data.

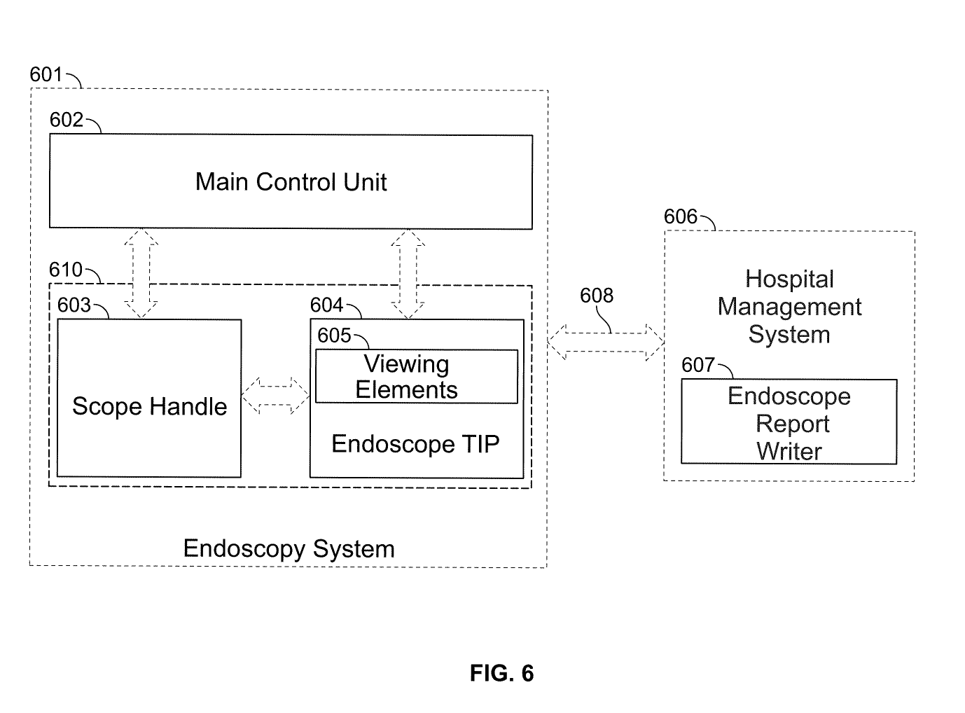

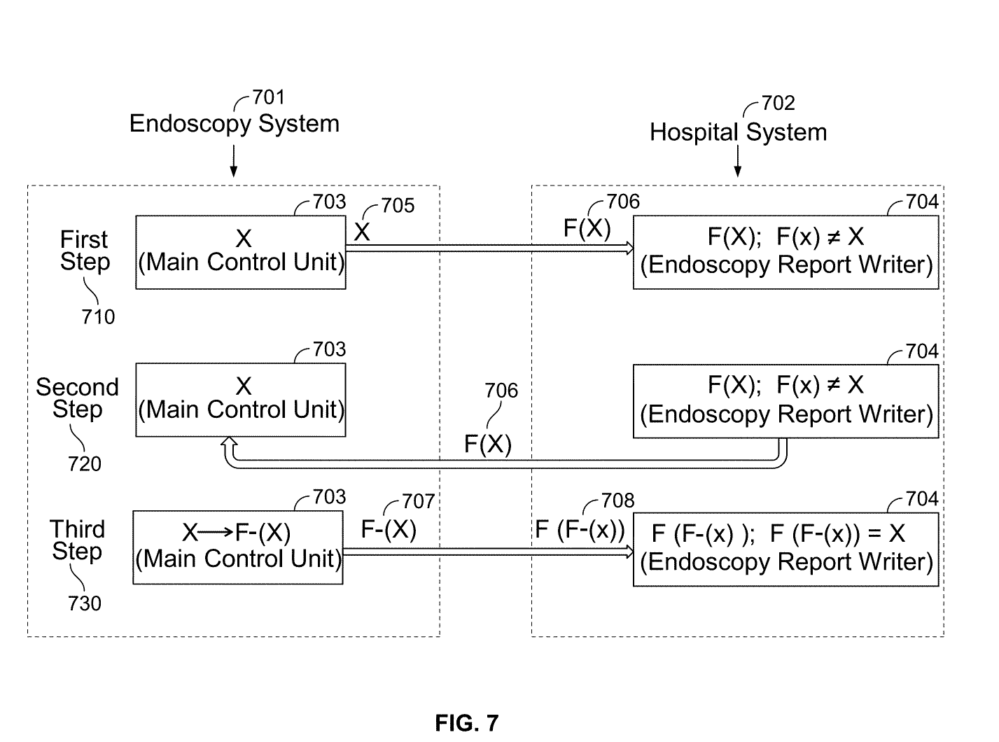

FIG. 7 is a flow diagram illustrating a method for pre-processing images and/or video in an endoscopy system in accordance with an embodiment of the present specification. As shown in FIG. 7, endoscopy system 701 comprises a main control unit 703 which is shown in data communication with the endoscopy report writer 704 associated with the hospital data management system 702. In one embodiment, the hospital data management system 702 is a software program which is used to manage multiple functions and the endoscopy report writer 704 is a specific module or software package included in the hospital data management system 702, and is used for endoscopy reporting functions. In one alternate embodiment, the endoscopy report writer 704 is a standalone software system developed for management, analysis and reporting of data received from an endoscope. The images captured by the viewing elements in the endoscopy system 701 are exported to the endoscopy report writer 704 through the main control unit 703.

As shown in FIG. 7, at step 710, the main control unit 703 transmits some image data X (represented as 705) and the same data is received as some function of X such as F(X) (represented as 706) by the endoscopy report writer (ERW) 704. In an ideal condition, the image data received by the endoscopy report writer 704 should be same as the image data sent by the main control unit 703 such that F(X)=X. However, in practical scenarios, because the data is transmitted from one system to another, there is often degradation of the quality of data and the data received at the endoscopy report writer 704 is not same; i.e. F(X)≠X. To determine the difference between the transmitted data X and the received data F(X), in one embodiment, the main control unit 703 stores a pre-defined set of image quality parameters and their corresponding acceptable threshold limits within which a deviation is allowed from the original transmitted data. The corresponding quality parameters in the image data received (back by the main control unit 703 from the endoscopy report writer 704) through a feedback control loop or system are compared with these threshold data limits to find any deviations beyond the respective threshold limits of the various quality parameters. In case any deviation is detected, the system assumes that F(X)≠X. One of ordinary skill in the art would appreciate that there could be multiple ways to control these threshold limits and the various quality parameters which are evaluated could vary depending on the specific requirement of each system.

There could be multiple ways in which the data quality is compromised during the transfer from one system to another. The image attributes and specifications may change because of which the image displayed in the final report generated by ERW system 704 may not reflect the actual image captured during the endoscopy procedure. In one embodiment of the present specification, a feedback control loop or mechanism is used to estimate the changes in image attributes and accordingly the image is preprocessed to reverse or offset these changes beforehand and therefore ensure that the actual image captured during the endoscopy process is received by the endoscopy report writer 704.

As illustrated in FIG. 7, at step 720, a feedback control system is shown, wherein the data corresponding to function F(X) (represented as 706) received by the endoscopy report writer 704 is sent to the main control unit 703. In one embodiment, the main control unit 703 compares the feedback data corresponding to F(X) with the original data X to estimate the function F(X). In one embodiment, the system uses predefined definitions and/or algorithms to compare the various parameters of the original image with corresponding parameters of the image received through the feedback system to estimate if there is a deviation and accordingly estimates the function F(X). In one embodiment the parameter X in the function F(X) may signify one or more of the various parameters such as color, contrast, hue, sharpness, black level, chroma or brightness of the image. Subsequently, once the function F(X) is estimated, at step 730, the main control unit 703 uses the function F(X) to modify the image data before exporting the data to the ERW system 704. In one embodiment, to reverse the impact of data degradation in the transmission process, instead of exporting the image data X, the main control unit 703, exports data corresponding to the function F⁻(X) (represented as 707) to the ERW system 704. In the above embodiment, when any data X is received by the ERW system 704, the data is modified by applying function F(X) to data X.

Once the main control unit 703 exports data corresponding to the function F⁻(X) to the ERW system 704, the data is modified such that function F(X) is applied on the incoming data, resulting in the ERW system 704 receiving the data as F(F⁻(X)) (represented as 708) which is the same as X. Therefore, by using a feedback control mechanism, the actual image quality is maintained while the image data is exported to an external ERW system.

In the above embodiment, the expected change in image attributes are pre-estimated and the image is modified before exporting it such that the image received at the ERW system 704 is same as the actual image captured during the endoscopy procedure. For example, in some cases, during transmission to ERW system 704, the brightness of the image may increase or decrease significantly making the image different from the actual image captured during the endoscopy procedure and difficult to interpret by a physician. During a case study, it was estimated that the brightness of the transmitted image increased or decreased by 25% upon transfer to the ERW system 704. Accordingly, the actual image captured during the endoscopy procedure was pre-processed to, respectively, reduce or increase brightness level by 20% by the main control unit 703 before transmission to the ERW system 704. Therefore, when the brightness of the transmitted image was again enhanced or degraded by 25% at the receptor ERW system 704, the actual image captured during the endoscopy procedure was retrieved.

In the above case scenario it was assumed that the percentage increase or decrease in brightness of the image during the transmission process is constant i.e. 25% and does not depend on the brightness level of the actual image captured during the endoscopy procedure. In cases where the percentage increase or decrease in brightness of the image is also a function of the brightness level of the original image, the percentage reduction or enhancement required in the brightness of the actual image is required to be adjusted accordingly.

It should be appreciated that brightness is a non-limiting exemplary parameter that is estimated and thereafter modulated. In various embodiments, other quality parameters such as color, contrast, hue, black level, sharpness, tone and/or chroma are pre-estimated and accordingly modulated by the main control unit 703 so that the image data received by the ERW system 704 is same as the original image data captured by the viewing elements of the endoscopy system 701. In various embodiments, reducing or increasing the quality parameters (color, contrast, hue, black level, sharpness, tone and/or chroma) of image data, before exporting the image data to the ERW system 704, in a range of 5% to 35% offsets or reverses the impact of increase or decrease 5% to 30% of the quality parameters due to the transmission of the image data to the ERW system 704.

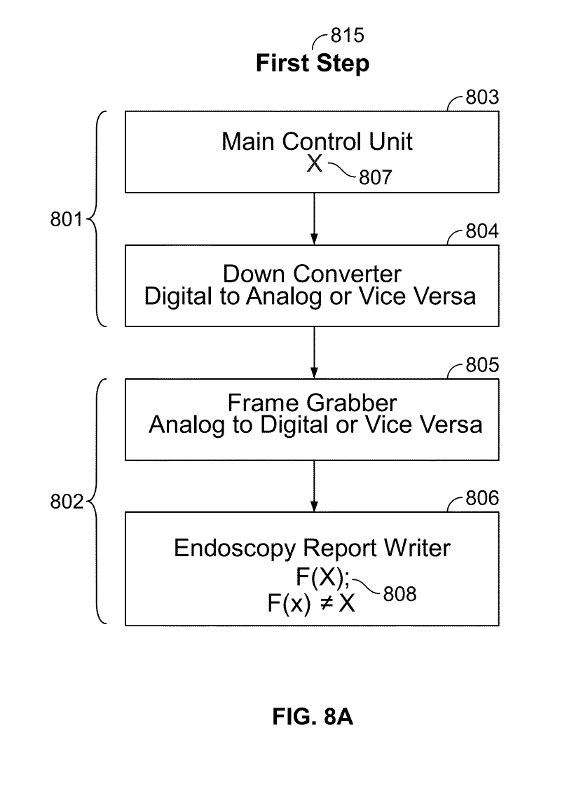

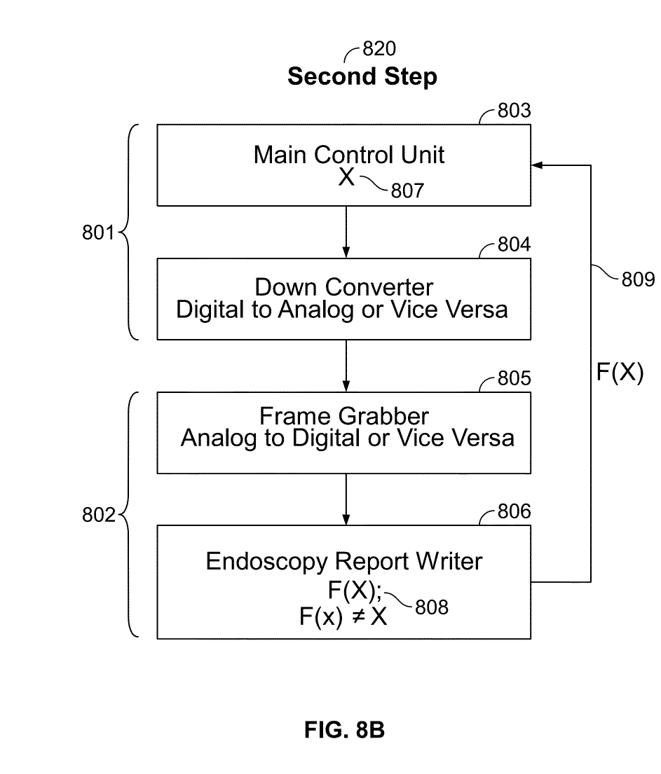

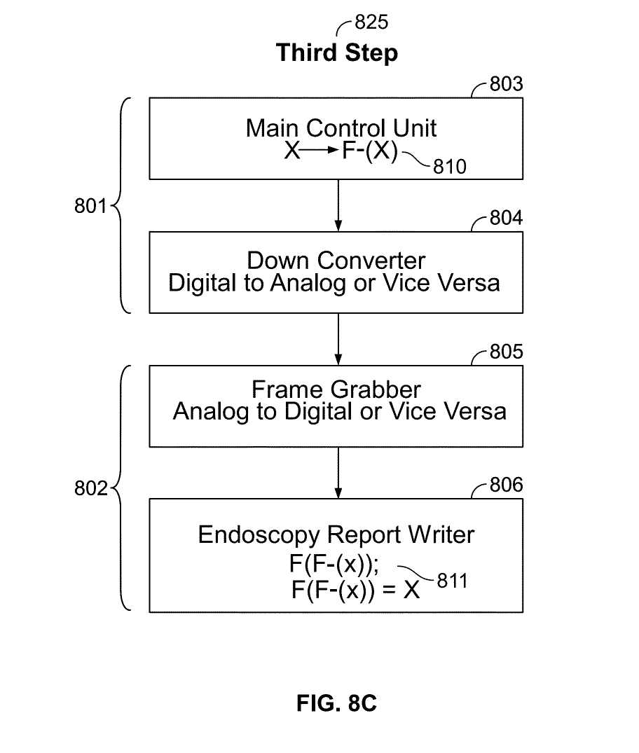

FIG. 8A illustrates a first step 815 of an image pre-processing method in accordance with an embodiment of the present specification. As shown in FIG. 8A, section 801 represents various components in an endoscopy system and section 802 represents various components in a hospital data management system. In one embodiment, the hospital data management system 802 comprises a documentation system such as an endoscopy report writer (ERW) program 806 typically used in hospitals for management, reporting and analysis of data received from an endoscopy system. The endoscopy system 801 comprises a multi-viewing elements endoscope (not shown) and a main control unit 803, which in an embodiment, contains or implement controls required for displaying images of internal organs captured by the endoscope on a display device. In one embodiment, the display device is configured to display images and/or video streams received from a plurality of viewing elements of the multi-viewing element endoscope.

In FIG. 8A, the images or video stream data transmitted by the main control unit 803 to the hospital data management system 802 is shown as X, (represented as 807). In one embodiment, the images or video stream displayed on the display devices are in digital format. In one embodiment, the endoscopy system 801, comprises a converter 804, which converts the images or video stream displayed on the display device from digital to analog format for transmission to external documentation systems such as the ERW system 806. In one embodiment, the hospital data management system 802 comprises a frame grabber 805 which receives data from the converter 804 in the endoscopy system 801. In an embodiment, the frame grabber 805 is a hardware or software based system that captures individual, digital still frames from an analog video signal or a digital video stream. It is typically employed as a component of a computer vision system, in which video frames are captured in digital form and then displayed, stored or transmitted further. In one embodiment, the frame grabber 805 captures digital frames from the incoming analog video signal received from the endoscope system 801.

Subsequently, the frame grabber 805 transmits the digital images or video stream to the endoscopy report writer system 806. The data transmission process from the endoscope system 801 to the hospital data management system 802 leads to a change in attributes or quality parameters of images or video signal. The process of data conversion from digital to analog format and subsequently from analog to digital format often causes the changes in image attributes. Therefore, the data received by the endoscopy report writer 806 is not exactly similar to the data X (represented as 807) actually transmitted by the endoscopy system 801. In one embodiment, the data received by endoscopy report writer 806 is some function of X such as F(X) (represented as 808 in FIG. 8A), wherein F(X) is not equal to X.

FIG. 8B illustrates a second step 820 of the image preprocessing method in accordance with an embodiment of the present specification. As the data transmission from the main control unit 803 to the endoscopy report writer 806 leads to some change or degradation in the image quality attributes or parameters, the data received at the endoscope report writer 806 is not equal to X (represented as 807 in FIG. 3B), but some function of X, such as F(X) (represented as 808 in FIG. 8B). As shown in FIG. 8B, once the data F(X) is received by the endoscopy report writer 806, in one embodiment, the same data F(X) is sent back to the main control unit 803 through a feedback control loop or system 809. In one embodiment, the transmission of data F(X) from the endoscopy report writer 806 to the main control unit 803 is done only during the initial set-up phase to estimate the image data quality attributes or parameters related changes that can occur during the transmission of the image data and subsequently, all data to be exported from the main control unit 803 is appropriately modulated to account for these changes before transmission. In one embodiment, the feedback control system 809 operates continuously and sends image and/or video streaming data to the main control unit 803 in real time. One of ordinary skill in the art would appreciate that there could be multiple methods for configuring the feedback control system 809.

In one embodiment, the feedback control system 809 operates through wireless transmission and in an alternate embodiment the feedback control system 809 is configured through physical wires. In one embodiment, the feedback control 809 is performed by manually retrieving the image data from the endoscopy report writer 806 on a memory device and manually providing this data to the main control unit 803 for comparison with original data. In one embodiment, the main control unit 803 compares the original image/video data X (represented as 807) with the data corresponding to function F(X) (represented as 808) received through the feedback control system 809, and estimates the mathematical function F(X) which is indicative of the data changes occurring during the transmission process.

FIG. 8C illustrates a third step 825 of the image preprocessing method in accordance with an embodiment of the present specification. As shown in FIG. 8C, to compensate for the changes in data that can occur during the transmission process, in one embodiment, the main control unit 803 modulates the data X (shown as 807 in FIG. 8B) by applying on the data the inverse of function F(X), estimated through the feedback control mechanism. Therefore, instead of exporting the data X, the main control unit exports the data F⁻(X) (represented as 810 in FIG. 8C), to the endoscopy report writer 806. The endoscopy report writer 806 receives any data Y as F(Y). Therefore, in this case, the endoscopy report writer 806 receives data F⁻(X) as F(F⁻(X)) (shown as 811 in FIG. 8C) which is equal to X. Therefore, by modifying the image/video data before transmitting it, the system nullifies the impact of changes that happen during the transmission process. The data corresponding to the actual image is received by the endoscopy report writer 806 and there is no degradation of the image quality.

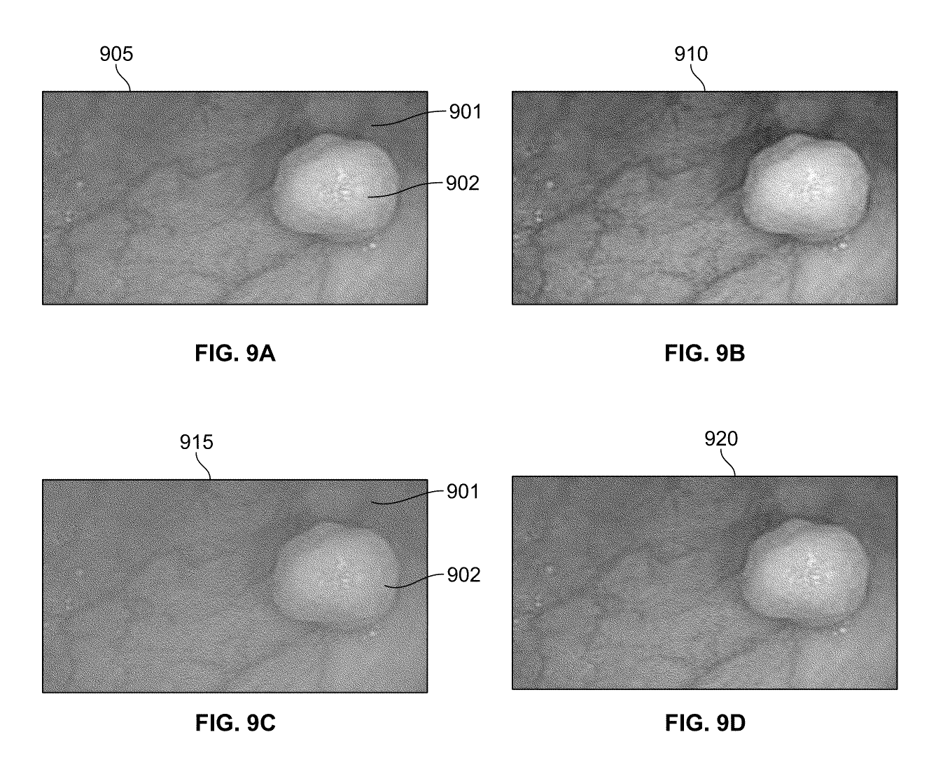

FIG. 9A illustrates an exemplary image 905 of an inside portion of a human body, such as a colon, captured through an endoscopy procedure. As shown in FIG. 9A, the image 905 comprises a lumen 901 and a polyp 902 present in the lumen 901 detected through an endoscopy procedure performed using an endoscopy system. In an embodiment, to enable preparation of a report on the endoscopy procedure, the imaging data 905 captured by the endoscopy system is transferred to a hospital data management system which comprises documentation systems such as an endoscopy report writer (ERW). When the image 905 is transferred from the endoscopy system to the external ERW system, the image attributes, parameters or specifications change because of which an image displayed in the report generated by the ERW system may not reflect the actual image 905 captured during the endoscopy procedure. The degradation in image quality may significantly reduce the reliability of medical procedure and make it difficult for a physician to accurately interpret the findings.

FIG. 9B illustrates one such exemplary image 910 received by the endoscopy report writer upon transmission of the original image 905 illustrated in FIG. 9A from the endoscopy system. As can be seen, the color contrast of the image 910 illustrated in FIG. 9B is significantly higher than that of the original image 905 (captured by the endoscopy system) shown in FIG. 9A. While, in the above example only the color contrast of the image 910 received at ERW is different from that of the original image 905, one of ordinary skill in the art may appreciate that multiple attributes (such as, but not limited to, hue, black level, sharpness, tone and/or chroma) of an image may be modified during the transfer of images from an endoscopy system to an ERW system.

Usually in such cases various image enhancement techniques are used to repair the modified image. In some case, a considerable time may have to be spent to fine tune the images. However, the image enhancement methods are usually unable to completely restore the image to the original form. The method disclosed in the present specification solves the above problem by enabling pre-processing of image/video data before transmitting the same from an endoscopy system to an external documentation system.

In one embodiment, the differences in the transmitted and the received images are determined by using a feedback control loop by a main control unit of the endoscope. Hence, the images 905 and 910 are compared by using the feedback control system disclosed in the present specification. Assuming that the color contrast data, attribute or parameter of the image 905 shown in FIG. 9A is defined as X and the color contrast increases by a specific percentage (say K %), the color contrast data of the received image 910 shown in FIG. 9B may be defined as F(X)=(1+K %)X. In one embodiment, the main control unit determines the function F(X) and modulates the original image data 905 by multiplying it with an inverse of function F(X) which is F⁻(X). In the above example, inverse function F⁻(X)=X/(1+K %). Therefore, the color contrast of the original image data 905 is reduced by multiplying it with 1/(1+K %) before transmission to the ERW.

It should be appreciated that in various embodiments, X may represent one or more of a plurality of quality attributes or parameters, such as color, contrast, hue, black level, sharpness, tone, chroma or brightness of the original image 905. If any one or more of these parameters changes (increases or decreases) by a specific percentage (say K %) the corresponding attributes or parameters of the received image 910 may be defined as F(X)=(1+K %)X (if the parameter X increases by K %) or F(X)=(1−K %)X (if the parameter X decreases by K %). In one embodiment, the main control unit determines the function F(X) and modulates the original image data 905 by multiplying it with an inverse of function F(X) which is F⁻(X). In accordance with the aforementioned example, inverse function F⁻(X)=X/(1+K %) (if the parameter X increases by K %) or F⁻(X)=X/(1−K %) (if the parameter X decreases by K %). Therefore, the affected or changes one or more attributes or parameters of the original image data 905 is modulated by multiplying it with 1/(1+K %) or 1/(1−K %) before transmission to the ERW.

FIG. 9C illustrates an exemplary modulated image 915 after the application of inverse mathematical function F⁻(X). As it can be seen in FIG. 9C the color contrast of the lumen 901 and the polyp 902 is lower than that of the image 905 illustrated in FIG. 9A, which is the actual image captured by the endoscope. In one embodiment, when the pre-processed image 915 shown in FIG. 9C is transmitted to the ERW, the color contrast of the image is enhanced by K % such that the color contrast of the final image 920 received by the ERW shown in FIG. 9D is same as that of the actual image 905 shown in FIG. 9A. FIG. 9D represents the final image 920 received by the ERW using the image pre-processing method disclosed in the present specification. As can be seen in the figures, the color contrast of the image 920 shown in FIG. 9D is same as that of the image 905 shown in FIG. 9A.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method of operating an endoscope comprising a main connector at a proximal end and an insertion section extending from the main connector towards a distal end, the main connector being operatively connected with a control unit, the method comprising:
storing operational information comprising usage information of one or more replaceable components of the endoscope in a first portion of a memory of the main connector;
storing manufacturing information comprising at least one manufacturing property of the endoscope in a second portion of the memory of the main connector, wherein the first portion of the memory is logically separated from the second portion of the memory;
retrieving the stored information; and
conveying the retrieved information.

2. The method of claim 1 further comprising storing operational information of the control unit in a memory of the control unit.

3. The method of claim 1 wherein storing operational information comprises storing at least one of an endoscope revision number, a date of last use of the endoscope, a usage information of one or more light sources, a cumulative number of procedures conducted using the endoscope, and a cumulative number of times the endoscope device is connected to the control unit.

4. The method of claim 1 wherein storing manufacturing information comprises storing at least one of a serial number of the endoscope, a type of the endoscope, and a type of video captured by the endoscope.

5. The method of claim 2 wherein storing operational information of the control unit comprises storing at least one of the cumulative operational time of the control unit, an operation time of the control unit during an endoscopy procedure, a number of times that the control unit is connected with the endoscope.

6. The method of claim 1 wherein storing operational information further comprises collecting usage information from at least one replaceable component of the endoscope device.

7. The method of claim 6 wherein collecting usage information comprises using Radio Frequency (RF) communication to obtain the usage information recorded in an RFID tag coupled with the replaceable component.

8. The method of claim 1 wherein conveying the retrieved information comprises displaying the retrieved information on a monitor connected to at least one of the control unit and the endoscope.

9. The method of claim 1 wherein conveying the retrieved information comprises communicating the retrieved information to a server operatively connected to the endoscope.

10. A method of operating an endoscope comprising a main connector at a proximal end and an insertion section extending from the main connector towards a distal end, the main connector being operatively connected with a control unit, the method comprising:
storing operational information of the control unit in a first memory device;
storing operational information comprising usage information of one or more replaceable components of the endoscope in a first portion of a second memory device;
storing manufacturing information comprising at least one manufacturing property of the endoscope in a second part of the second memory device;
retrieving the stored information; and,
conveying the retrieved information.

11. The method of claim 10 wherein storing operational information of the control unit comprises storing at least one of the cumulative operational time of the control unit, an operation time of the control unit during an endoscopy procedure, a number of times that the control unit is connected with the endoscope.

12. The method of claim 10 wherein operational information comprising usage information of one or more replaceable components of the endoscope comprises storing at least one of an endoscope revision number, a date of last use of the endoscope, a usage information of one or more light sources, a cumulative number of procedures conducted using the endoscope, and a cumulative number of times the endoscope device is connected to the control unit.

13. The method of claim 10 wherein storing manufacturing information of the endoscope device comprises storing at least one of a serial number of the endoscope, a type of the endoscope, and a type of video captured by the endoscope.

14. The method of claim 10 wherein storing operational information comprising usage information of one or more replaceable components of the endoscope further comprises collecting operational information from at least one replaceable component of the endoscope device.

15. The method of claim 14 wherein collecting usage information comprises using Radio Frequency (RF) communication to obtain the usage information recorded in an RFID tag coupled with the replaceable component.

16. The method of claim 10 wherein conveying retrieved information comprises displaying retrieved information on a monitor connected to at least one of the control unit and the endoscope device.

17. The method of claim 10 wherein conveying the retrieved information comprises communicating the retrieved information to a server operatively connected to the endoscope.

18. The method of claim 1 wherein when the endoscope is connected to the control unit, the control unit is adapted to detect the connection and cause a memory counter to be updated, wherein said memory counter is configured to track a cumulative number of times the endoscope is plugged to the control unit.

19. The method of claim 1 wherein when the endoscope is connected to the control unit, the control unit is adapted to detect the connection and cause a memory counter to be updated to a new date, wherein said memory counter is configured to track a last usage date of the endoscope.

20. The method of claim 1 wherein the endoscope further comprises a plurality of illuminators and wherein, when at least one of said plurality of illuminators is switched on, the control unit is adapted to send a signal to a memory counter, wherein said memory counter is configured to track a number of endoscopy procedures performed and a cumulative number of times each of said plurality of illuminators were switched on or off.

21. The method of claim 1 wherein the endoscope further comprises a plurality of illuminators and wherein, when at least one of said plurality of illuminators is switched off, the control unit is adapted to send a signal to a memory counter, wherein said memory counter is configured to track a number of endoscopy procedures performed and a cumulative number of times each of said plurality of illuminators were switched on or off.

22. The method of claim 1 wherein when the endoscope is disconnected from the control unit, the control unit is adapted to detect the disconnection and cause a memory counter to be updated, wherein said memory counter is configured to track a total duration for which the endoscope remained plugged into the control unit.

23. The method of claim 1 wherein the control unit is adapted to generate at least one of an average duration for a single procedure over a predefined period of time, a longest duration for single procedure, a shortest duration for a single procedure, and an average duration of use for a single procedure on a per physician basis.
